(19) United States
(12) Patent Application Publication
(10) Pub. No.: US 2009/0012016 A1
(43) Pub. Date: Jan. 8, 2009

(54) Short Interfering Rna and Micro-Rna Compounds and Methods of Designing, Making, and Using the Same

(76) Inventors: Zissimos Mourelatos, Philadelphia, PA (US); Anup Sharma, Philadelphia, PA (US); Marianthi Kiriakidou, Philadelphia, PA (US)

Correspondence Address: Pepper Hamilton LLP, 400 Berwyn Park, 899 Cassatt Road, Berwyn, PA, 19312-1183, US

(21) Appl. No.: 10/576,481

(22) PCT Filed: Oct. 22, 2004
(86) PCT No.: PCT/US04/35124

§ 371 Date: May 15, 2008

Related U.S. Application Data
(60) Provisional application No. 60/513,489, filed on Oct. 22, 2003.

(51) Int. Cl. A61K 31/7105; C40B 30/02; A61P 43/00; C07H 21/02

(52) U.S. Cl. 514/44; 506/8; 536/24.5

(57) ABSTRACT

Methods of identifying, designing and synthesizing uniquely targeting siRNA nucleotide sequences for a target mRNA sequence of a target species are disclosed. Methods of identifying, designing and synthesizing miRNA nucleotide sequences that does not function as a siRNA nucleotide sequence for mRNA of a target species are disclosed. Method of inhibiting expression of a target mRNA molecule are disclosed sequence. siRNA molecules including uniquely targeting siRNA molecules and RISCs comprising the same are disclosed. miRNA molecules that do not function as siRNA nucleotide sequences for mRNA of a target species are disclosed.

### FIELD OF THE INVENTION

The present invention relates to siRNA and miRNA compounds and methods of designing, synthesizing and using them to inhibit production of protein by inhibiting mRNA translation.

### BACKGROUND OF THE INVENTION

An evolutionary conserved mechanism of gene expression regulation, based on small, noncoding RNAs, exists in diverse organisms from Schizosaccharomyces pombe (1) to humans (2) (3). A class of ˜22 nucleotide (nt) small RNAs, termed short interfering RNAs (siRNAs) (4) (5) are critical determinants of RNA interference (RNAi), an RNA-based silencing mechanism (6), whose physiological function includes protecting the genome against invading nucleic acids such as viruses and transposons (7) (8).

In RNAi, double-stranded RNA (dsRNA) is converted by Dicer, an RNAse III-type nuclease (9), into siRNA duplexes that incorporate in ribonucleoprotein complexes (RNPs) termed RNA-induced silencing complexes (RISCs) (10). Unwinding of siRNA duplexes activates RISC and enables it to recognize target RNAs that are complementary to the guide strand of the siRNA (11). An unknown endonuclease(s) present in RISC cleaves the target RNA across from the center of the guide siRNA (5) (11).

Another class of small ˜22 nt RNAs, termed microRNAs (miRNAs) (2) (12) (13) (3) are processed by Dicer (14) (15) (16) from the stems of longer (˜75 nt) RNA precursors (pre-miRNAs) that form stem-loop structures. miRNAs incorporate in microRNPs (miRNPs) along with at least three proteins: eukaryotic translation Initiation Factor 2C2 (eIF2C2), a member of the Argonaute family of proteins; Gemin3, a putative RNA helicase; and Gemin4 (3). Animal miRNAs recognize sequences with partial antisense complementarity in the 3′ untranslated regions of their target mRNAs and prevent the accumulation of nascent polypeptides (17) (18) (19) (20).

Argonaute proteins (21) are essential for RNAi and play critical roles in the maturation and function of si/miRNAs (22) (23) (24) (25) and are also present in Drosophila (10) and human RISC complexes (26); their biochemical functions however, are unknown. Hutvagner and Zamore have recently made the important observation that RISC and miRNPs are very similar complexes because immunopurified miRNPs containing let-7, an endogenous miRNA, are able to cleave a target RNA that contains a fully complementary site to let-7 (27). Furthermore, plant miRNAs with extensive complementarity to their target mRNAs act as siRNAs by cleaving their mRNA targets (28) (29) (30). These findings indicate that a miRNA-directed endonuclease, similar to the siRNA-directed RISC endonuclease, is present in miRNPs.

Since the discovery of RNAi, much effort has been directed toward using RNAi to control gene expression, such as in the treatment of disease and infection. For example, RNAi is proposed for use in treatment of cancer by post transcriptional inhibition of oncogene expression. Other targets for RNAi post translational inhibition of gene expression include other disease causing genes or genes whose expression causes undesirable effects or symptoms. RNAi has also been proposed to inhibit gene expression of pathogen genes such as viral genes.

To use RNAi in the treatment of disease and infection, siRNA or miRNA are designed based upon the nucleotide sequence of the mRNA that is targeted for post transcriptional inhibition of expression. An siRNA or miRNA so designed may not only inhibit the targeted mRNA but other mRNA as well. This lack of specificity can cause unwanted side effects by inhibiting expression of genes whose expression is necessary.

There remains a need for improvements in the methods of designing, synthesizing and use of siRNAs and miRNAs. There is a need for methods of determining whether an siRNA or miRNA will be target specific. There is a need for improved siRNAs and miRNAs.

### SUMMARY OF THE INVENTION

The present invention relates to methods of identifying a uniquely targeting siRNA nucleotide sequence for a target mRNA sequence of a target species. The methods comprise the steps of: comparing a database of mRNA sequences from the target species with an siRNA nucleotide sequence that consists of 18-25 including at least 11 consecutive nucleotides complementary to the target mRNA sequence to be cleaved by the siRNA nucleotides, wherein the at least 11 consecutive nucleotides complementary to the target mRNA sequence include a nucleotide that is third from an siRNA nucleotide sequence's 5′ end; and determining if, in addition to the target mRNA sequence, one or more additional mRNA sequences in the database are complementary to an 11 consecutive nucleotide sequence of the siRNA nucleotide sequence including the third nucleotide from the 5′ end of the siRNA nucleotide. An absence of one or more additional mRNA sequences in the database that are complementary to an 11 consecutive nucleotide sequence of the siRNA nucleotide sequence including the third nucleotide from the 5′ end of the siRNA nucleotide indicates that the siRNA nucleotide sequence is a uniquely targeting siRNA nucleotide sequence.

The present invention further relates to methods of designing a uniquely targeting siRNA for a target mRNA molecule. The methods comprise the steps of identifying an siRNA nucleotide sequence for the target mRNA, said sequence consisting of 18-24 nucleotides including a nucleotide sequence that has 11 consecutive nucleotides, including the third nucleotide from the siRNA nucleotide sequence's 5′ end, that are complementary to an 11 nucleotide sequence that occurs on the target mRNA molecule; comparing the siRNA nucleotide sequence with a database of mRNA sequences from the target mRNA species; and determining if, in addition to the target mRNA sequence, one or more additional mRNA sequences in the database are complementary to an 11 consecutive nucleotide sequence of the siRNA nucleotide sequence including the third nucleotide from the 5′ end of the siRNA nucleotide. An absence of one or more additional mRNA sequences in the database that are complementary to an 11 consecutive nucleotide sequence of the siRNA nucleotide sequence including the third nucleotide from the 5′ end of the siRNA nucleotide indicates that the of the siRNA nucleotide is a uniquely targeting siRNA nucleotide sequence.

The present invention further relates to methods of synthesizing a uniquely targeting siRNA for a target mRNA molecule. The methods comprise the steps of: identifying or designing uniquely targeting siRNA nucleotide sequence for the target mRNA according to any of claims 1 to 12; and synthesizing an siRNA molecule having the uniquely targeting siRNA nucleotide sequence.

The present invention further relates to a method of inhibiting expression of a target mRNA molecule comprising the steps of: synthesizing a uniquely targeting siRNA for a target mRNA molecule and contacting a target mRNA molecule with an RISC that comprises an siRNA molecule that comprises the uniquely targeting siRNA nucleotide sequence.

The present invention further relates to methods of identifying an miRNA nucleotide sequence that does not function as a siRNA nucleotide sequence for mRNA of a target species comprising the steps of: comparing a database of mRNA sequences from the target species with an miRNA nucleotide sequence that consists of 18-24 nucleotides; and determining if one or more mRNA sequences in the database are complementary to an 11 consecutive nucleotide sequence of the miRNA nucleotide sequence including the third nucleotide from the 5′ end of the miRNA nucleotide. An absence of one or more mRNA sequences in the database that are complementary to an 11 consecutive nucleotide sequence of the miRNA nucleotide sequence including the third nucleotide from the 5′ end of the miRNA nucleotide indicates that the miRNA nucleotide sequence does not function as a siRNA nucleotide sequence for mRNA of a target species.

The present invention further relates to methods of designing an miRNA nucleotide sequence that does not function as a siRNA nucleotide sequence for mRNA of a target species. The methods comprises the steps of: identifying an miRNA nucleotide sequence that consists of 18-24 nucleotides; comparing the miRNA nucleotide sequence with a database of mRNA sequences from the target mRNA species; and determining if one or more mRNA sequences in the database are complementary to an 11 consecutive nucleotide sequence of the miRNA nucleotide sequence including the third nucleotide from the 5′ end of the miRNA nucleotide. An absence of one or more mRNA sequences in the database that are complementary to an 11 consecutive nucleotide sequence of the miRNA nucleotide sequence including the third nucleotide from the 5′ end of the siRNA nucleotide indicates that the miRNA nucleotide sequence does not function as a siRNA nucleotide sequence for mRNA of a target species.

The present invention further relates to methods of synthesizing an miRNA that does not function as a siRNA nucleotide sequence for mRNA of a target species. The methods comprise the steps of: identifying or designing an miRNA nucleotide sequence that does not function as an siRNA nucleotide sequence for mRNA of a target species according to any of claims 15 to 26; and synthesizing an miRNA molecule that has an miRNA nucleotide sequence that does not function as an siRNA nucleotide sequence for mRNA of a target species.

The present invention further relates to methods of inhibiting expression of a target mRNA molecule. The methods comprise the steps of: synthesizing an miRNA that does not function as a siRNA nucleotide sequence for mRNA of a target species and contacting a target mRNA molecule with an miRNP that comprises an miRNA that does not function as a siRNA nucleotide sequence for mRNA of a target species.

The present invention further relates to uniquely targeting siRNA molecule consisting of 18-25 nucleotides including a nucleotide sequence that has at least 11 consecutive nucleotides, including the third nucleotide from the 5′ end of the siRNA molecule, that are complementary to a nucleotide sequence that occurs on a target mRNA molecule and not on another mRNA from a target species and RISCs comprising same.

The present invention further relates to siRNA molecules consisting of 18-25 nucleotides including a nucleotide sequence that has at least 11 consecutive nucleotides, including the third nucleotide from the 5′ end of the siRNA molecule, that are complementary to a nucleotide sequence that occurs on a target mRNA molecule and not on another mRNA from a target species, wherein the siRNA molecule is not fully complementary with a sequence of the target mRNA molecule and RISCs comprising same.

The present invention further relates to miRNA molecules for a target mRNA of a target species, wherein said miRNA molecule consists of 18-25 nucleotides and does not function as a siRNA nucleotide sequence for mRNA of a target species.

### BRIEF DESCRIPTION OF THE FIGURES

FIGS. 1A-1D show data from the characterization of purified eIF2C2 protein and associated miRNAs. In FIG. 1A, aliquots from indicated eluates were resolved on a 4-12% NuPAGE gel and visualized by silver staining. Arrow indicates 0.4 μg of purified human eIF2C2 protein. Molecular mass markers (in kilodaltons) is shown on the left. In FIG. 1B, RNA was isolated from total HeLa cell lysate (T), from 8C7 eluate containing 100 ng of immunopurified eIF2C2 protein and from an equivalent volume of control IgG eluate; the RNA was 3′-end labeled with [5′-³²P]-pCp and was resolved by electrophoresis on a 10% denaturing polyacrylamide gel. Nucleotide sizes of the radiolabeled marker (M) are shown on the left. In FIG. 1C, RNA isolated from 8C7 eluate, containing 5 pmoles of purified eIF2C2, along with the indicated amounts of a synthetic let-7a RNA, was analyzed by Northern hybridization using a radiolabeled oligonucleotide probe complementary to let-7a. In FIG. 1D, quantitation of C; black circles: synthetic let-7; open square: let-7 from 8C7 eluate.

FIGS. 2A-2C show that the eIF2C2/let7a-miRNA ribonucleoprotein (miRgonaute) is a miRNP/RISC endonuclease. FIG. 2A shows a schematic of the RNA targets used (designated let7TI and let7-TP) and the potential base pairing with let-7a; the [5′-³²P] of pCp is shown. Cleavage sites are indicated with lightning bolt. In FIG. 2B, the indicated, 3′-end radiolabeled RNA targets were incubated with purified let-7-containing eIF2C2 (eIF2C2/let-7) or with IgG eluate. The products of the reactions were analyzed on 15% denaturing polyacrylamide gels. In FIG. 2C, 3′-end labeled let7-TP RNA target was incubated with purified eIF2C2/let-7a for the times indicated either with or without ATP or Mg⁺⁺ ions (as shown), and were analyzed as in FIG. 2B. Nucleotide sizes of the radiolabeled marker (M) are shown on the left. The RNA targets are indicated by brackets and the 3′-cleavage products by arrows.

FIGS. 3A-3F relate to target RNA-miRNA pairing requirements for miRgonaute mediated cleavage. FIGS. 3A, 3C and 3E show schematic of the RNA targets used (designated let7TA, let7-TB, let7TC and let7-TD) and the potential base pairing with let-7a; the ³²P radiolabel is shown as p. Lightning bolts indicate cleavage sites. In FIGS. 3B, 3D and 3F, the RNA targets were incubated with (+) or without (−) purified let-7a-containing-eIF2C2 (eIF2C2/let-7a) and the products of the reaction were analyzed on a 20% denaturing polyacrylamide gel. The cleavage products are indicated with arrows.

FIGS. 4A and 4B shows the minimal requirements for target RNA recognition and cleavage by mi/siRNAs. FIG. 4A shows a schematic of a miRgonaute ribonucleoprotein bound to its cognate mRNA target. miRNA nucleotides that are critical for target recognition and cleavage are shown base paired with the target RNA nucleotides. Other, as yet unidentified, factor(s) (Factor X) might also contribute to target RNA recognition and cleavage, in vivo. FIG. 4B shows a schematic of a mi/siRNA bound to its target. The stretch of the critical nucleotides of an mi/siRNA required for target RNA recognition and cleavage are indicated (nucleotides between the two arrows). To design siRNAs with high specificity these nucleotides should not base pair with any mRNA but the one to be targeted. Cleavage site of the target RNA is indicated with a lightning bolt.

### DETAILED DESCRIPTION OF PREFERRED EMBODIMENTS

The discovery that less than complete complementarity is required between an siRNA sequence and target RNA in order for RISC to function provides the opportunity to undertake several procedures to improve applications of RNAi technology as well as providing the basis for uniquely targeted siRNA compounds. Improved applications include 1) methods of determining whether or not an siRNA sequence uniquely targets a target RNA sequence, 2) improved methods of designing siRNA sequences, 3) improved methods of synthesizing siRNA sequences, and 4) improved methods of inhibiting gene expression. The discovery also provides the basis for siRNA compounds that uniquely target a target RNA and siRNA compounds that are functional siRNA compounds despite lacking complete complementarity to a target RNA sequences.

As used herein, the term “uniquely targeting siRNA” is meant to refer to an siRNA which will only be functional as part of a RISC to eliminate a target RNA and will not eliminate a different RNA present, such as a different RNA from the same species as that of the target RNA.

An siRNA will be functional as part of a RISC, i.e. it will eliminate a target RNA, if eleven consecutive nucleotides of its sequence, including the third nucleotide from the 5′ end are completely complementary to those of the target RNA.

An siRNA sequence can be 18-25 nucleotides, and preferably about 22 nucleotides. An siRNA is generated by the processing of double stranded RNA (dsRNA) sequences of a larger RNA molecule by the enzyme DICER into siRNA duplexes which are short double stranded RNAs with each strand having 18-25 nucleotides. Generally, the siRNA duplex has a two nucleotide overhang on each end. The siRNA together with certain proteins forms a ribonucleoprotein complex which is referred to as a RISC. The siRNA ceases to be a duplex in an active RISC. When the single stranded siRNA of the RISC hybridizes with a target RNA, the RISC functions to degrade the target RNA.

The degree of complementarity between a single stranded siRNA of the RISC and a target RNA required for the RISC to degrade the target RNA is less than complete. As used herein “the rule” is meant to refer to the requirement that a single stranded siRNA of the RISC have a sequence of at least eleven consecutive nucleotides that are fully complementary with an eleven nucleotide sequence of the target RNA and that the at least eleven consecutive nucleotides of the siRNA include the third nucleotide from the 5′ end of the siRNA. Accordingly, for a siRNA with 22 nucleotides, to fit the rule, full complementarity of the siRNA sequence with a target RNA sequence of the following siRNA nucleotides is sufficient to produce a functional RISC: 1-11, 1-12, 1-13, 1-14, 1-15, 1-16, 1-17, 1-18, 1-19, 1-20, 1-21, 1-22, 2-12, 2-13, 2-14, 2-15, 2-16, 2-17, 2-18, 2-19, 2-20, 2-21, 2-22, 3-13, 3-14, 3-15, 3-16, 3-17, 3-18, 3-19, 3-20, 3-21 and 3-22. For a siRNA with 18 nucleotides, to fit the rule, full complementarity of the siRNA sequence with a target RNA sequence of the following siRNA nucleotides is sufficient to produce a functional RISC: 1-11, 1-12, 1-13, 1-14, 1-15, 1-16, 1-17, 1-18, 2-12, 2-13, 2-14, 2-15, 2-16, 2-17, 2-18, 3-13, 3-14, 3-15, 3-16, 3-17 and 3-18. For a siRNA with 19 nucleotides, to fit the rule, full complementarity of the siRNA sequence with a target RNA sequence of the following siRNA nucleotides is sufficient to produce a functional RISC: 1-11, 1-12, 1-13, 1-14, 1-15, 1-16, 1-17, 1-18, 1-19, 2-12, 2-13, 2-14, 2-15, 2-16, 2-17, 2-18, 2-19, 3-13, 3-14, 3-15, 3-16, 3-17, 3-18 and 3-19. For a siRNA with 20 nucleotides, to fit the rule, full complementarity of the siRNA sequence with a target RNA sequence of the following siRNA nucleotides is sufficient to produce a functional RISC: 1-11, 1-12, 1-13, 1-14, 1-15, 1-16, 1-17, 1-18, 1-19, 1-20, 2-13, 2-14, 2-15, 2-16, 2-17, 2-18, 2-19, 2-20, 3-13, 3-14, 3-15, 3-16, 3-17, 3-18, 3-19, and 3-20. For a siRNA with 21 nucleotides, to fit the rule, full complementarity of the siRNA sequence with a target RNA sequence of the following siRNA nucleotides is sufficient to produce a functional RISC: 1-11, 1-12, 1-13, 1-14, 1-15, 1-16, 1-17, 1-18, 1-19, 1-20, 1-21, 2-12, 2-13, 2-14, 2-15, 2-16, 2-17, 2-18, 2-19, 2-20, 2-21, 3-13, 3-14, 3-15, 3-16, 3-17, 3-18, 3-19, 3-20, and 3-21. For a siRNA with 23 nucleotides, to fit the rule, full complementarity of the siRNA sequence with a target RNA sequence of the following siRNA nucleotides is sufficient to produce a functional RISC: 1-11, 1-12, 1-13, 1-14, 1-15, 1-16, 1-17, 1-18, 1-19, 1-20, 1-21, 1-22, 1-23, 2-12, 2-13, 2-14, 2-15, 2-16, 2-17, 2-18, 2-19, 2-20, 2-21, 2-22, 2-23, 3-13, 3-14, 3-15, 3-16, 3-17, 3-18, 3-19, 3-20, 3-21, 3-22 and 3-23. For a siRNA with 24 nucleotides, to fit the rule, full complementarity of the siRNA sequence with a target RNA sequence of the following siRNA nucleotides is sufficient to produce a functional RISC: 1-11, 1-12, 1-13, 1-14, I-15, 1-16, 1-17, 1-18, 1-19, 1-20, 1-21, 1-22, 1-23, 1-24, 2-12, 2-13, 2-14, 2-15, 2-16, 2-17, 2-18, 2-19, 2-20, 2-21, 2-22, 2-23, 2-24, 3-13, 3-14, 3-15, 3-16, 3-17, 3-18, 3-19, 3-20, 3-21, 3-22, 3-23 and 3-24. For a siRNA with 25 nucleotides, to fit the rule, full complementarity of the siRNA sequence with a target RNA sequence of the following siRNA nucleotides is sufficient to produce a functional RISC: 1-11, 1-12, 1-13, 1-14, 1-15, 1-16, 1-17, 1-18, 1-19, 1-20, 1-21, 1-22, 1-23, 1-24, 1-25, 2-12, 2-13, 2-14, 2-15, 2-16, 2-17, 2-18, 2-19, 2-20, 2-21, 2-22, 2-23, 2-24, 2-25, 3-13, 3-14, 3-15, 3-16, 3-17, 3-18, 3-19, 3-20, 3-21, 3-22, 3-23, 3-24 and 3-25. Those where the complementary begins at nucleotide four from the 5′ end or further 5′ will not function, i.e. they do not fit the rule.

It is possible to determine whether or not a given sequence fits the rule for known RNA sequences. In a preferred embodiment, this can be done in an automated fashion using software which is or can be programmed to test for the rule using a given sequence and a database of known RNA sequences. Generally, the database is to test a sequence against known RNA sequences from the same species. In some embodiments, the search algorithm is the BLAST algorithm which is readily obtainable and in the public domain. In some embodiments, the mRNA databases are selected from the database deposited with NCBI and the database deposited with ENSEMBL. Both databases are readily accessible and in the public domain.

In some embodiments, a particular siRNA sequence or miRNA sequence is known and compared with RNA sequences to determine if the siRNA sequence will function with an RNA other than the target RNA, i.e. determine whether it uniquely targets the target RNA or if it will function against non-target RNA as well. In the case of testing an siRNA sequence, this information provides insight into whether or not a given siRNA will function beyond processing the desired target RNA. In the case of testing an miRNA sequence, this information provides insight into whether or not a given miRNA can function as an active siRNA against a target beyond functioning to inhibit translation of the desired target RNA. In either case, the RNAi sequence can be compared with known RNA sequences, preferably using a computer [program to test against a database of RNA sequences, to determine if any unintended activity will result. The ability to test for predicted unintended activity allows for a number of candidate siRNA or miRNA sequences to be analyzed to select for those which will not have unintended activity.

In some embodiments, a particular RNA sequence can be identified as one to be targeted for siRNA mediated degradation and using the target RNA sequence a uniquely targeted siRNA sequence can be designed. Sequences of 18-25 nucleotides can be tested to determine if those which fit the rule will not have unintended activity. Importantly, while not limited to eleven consecutive nucleotides including the third from the 5′ end, only those eleven must have full complementarity. The remaining 7-14 nucleotides can independently be complementary or not. A computer can be programmed to use the sequence of the target RNA to generate nucleic acid sequences of 18-25 which fit the rule and are unique when compared to a database of known RNA sequences. In some embodiments, such a program may initially test 18-25 nucleotide fragments of the target RNA to determine such fragments with full complementarity to the target RNA fit the rule for any non-target RNA sequences. In some embodiments, such a program may sequences of 18-25 nucleotides that fit the rule for the target RNA and determine if such sequences fit the rule for any non-target RNA sequences.

Once a sequence has been determined to be a uniquely targeting siRNA for a particular target RNA, the sequence can be used to synthesize an active siRNA system. Commercially available kits may be employed to generate an active siRNA system for a given siRNA sequence. Such kits may be obtained from various commercial sources including Ambion, Inc. (Austin, Tex.); IMGENEX Corporation (San Diego, Calif.), InvivoGen (San Diego, Calif.); Mirus (Madison, Wis.), New England Biolabs (Beverly, Mass.); Promega (Madison, Wis.); QIAGEN (Venlo, the Netherlands); and Spring Bioscience (Fremont, Calif.). Similarly, once it has been determined that an miRNA sequence will not target an RNA as a functional siRNA, the miRNA can be synthesized.

Uniquely targeting siRNA for a particular target RNA may be used to inhibit expression of the target gene by eliminating the target mRNA, thereby preventing prior to translation and protein production. Functional miRNA for a particular target RNA may be used to inhibit expression of the target gene by interfering with translation of the target mRNA, and thus protein production. When the miRNA sequence cannot generate an active siRNA sequence no unintended siRNA activity will be associated with use of the miRNA.

In some embodiments, a particular siRNA sequence or miRNA sequence is known and compared with RNA sequences to determine if the siRNA sequence will function with an RNA other than the target RNA, i.e. determine whether it uniquely targets the target RNA or if it will function against non-target RNA as well. In the case of testing an siRNA sequence, this information provides insight into whether or not a given siRNA will function beyond processing the desired target RNA. In the case of testing an miRNA sequence, this information provides insight into whether or not a given miRNA can function as an active siRNA against a target beyond functioning to inhibit translation of the desired target RNA. In either case, the RNAi sequence can be compared with known RNA sequences, preferably using a computer [program to test against a database of RNA sequences, to determine if any unintended activity will result. The ability to test for predicted unintended activity allows for a number of candidate siRNA or miRNA sequences to be analyzed to select for those which will not have unintended activity.

In some embodiments, siRNA compounds and RISCs comprising such siRNA compounds are provided in which the siRNA sequence is a uniquely targeting siRNA sequence. In some preferred embodiments, the uniquely targeting siRNA and the RISCs comprising the same are directed against RNA encoding oncogenes, tumor suppressor genes, genes causing neurogenerative diseases, viral genes, cytokines, interleukins, chemokines, co-stimulatory molecules and growth factors. In some preferred embodiments, the uniquely targeting siRNA and the RISCs comprising the same are directed against RNA encoding p53, MYC, MYB, PTEN, HER-2/NEU, TGF, PDGFR, Ras, Rb (Retinoblastoma protein), bcl-2, bax, phosphatase 2A, telomerase, p16, p21, Cyclin Dependent Kinases (CDKs), TRAIL, TNF, Mitogen Activated Kinases (MAPKs), VHL, NfkB, Merlin, Schwanomin, Notch, APC, AXIN2, E-Cadherin, GPC3, AXT1,2. Patched, SUFU, FH, SDHB,C,D, WT-1, STK11, TSC1, TSC2, BMPR1A, SMAD4, NF2, BHD, HRPT2, MUTYH, ATM, BLM, BRCA1, BRCA2, FANCA,C,D2,E,F,G, MSH2, MLH1, MSH6, PMS2, XPA, KT, MET, RET, HIF-1, b-catenin, FAS, FBXW7, GLI, HPEVE6, MDM2, AKT2, FOXO1A,3A, PI3KCA, CYCLIN D1, HPVE7, TAL1, TFE3, ALK, ABL1, BRAF, EGFR, EPHB2, ERBB2, FES, FGFRs, FLT3,4, JAK2, N-RAS, K-RAS, PDGFB, PDGFRB, EWSR1, RUNX1, SMAD2, TGFFBR1, 2, MYCN, HMGA2, HOXA9,11,13, HOX11,11L2, MKK4, MLL, SS18, RARA, PTNP1,11, APP (Amyloid Precursor protein), Tau, synucleins, alpha-synuclein (PARK1), Parkin (PARK2), PARK5, Huntingtin, SOD (Superoxide Dismutase), Senataxin, Apolipoprotein-E, DJ-1 (PARL7), presenilin-1, presenilin-2, IL-1, IL-2, IL-4. TNF, Interferons, (IFNA, IFNB, IFN-gamma), CD4, CD8, CD19, CD5, CD43, CD1, CD3, B-cell receptor, T-cell Receptor, VEGF, bcr/abl, HIV genes such asrev, env, gag, or CMV genes.

In some embodiments, siRNA compounds and RISCs comprising siRNA compounds are provided in which the siRNA sequence is less than fully complementary with a target RNA sequence. These siRNA sequences fit the rule but are less than completely complementary. In some embodiments, 11, 12, 13, 14, 15, 16, 17 or 18, (including the third from the 5′ end) of 18 nucleotides are complementary with a sequence of the target RNA. In some embodiments, 11, 12, 13, 14, 15, 16, 17, 18 or 19 (including the third from the 5′ end) of 19 nucleotides are complementary with a sequence of the target RNA. In some embodiments, 11, 12, 13, 14, 15, 16, 17, 18, 19 or 20 (including the third from the 5′ end) of 20 nucleotides are complementary with a sequence of the target RNA. In some embodiments, 11, 12, 13, 14, 15, 16, 17, 18, 19, 20 or 21 (including the third from the 5′ end) of 21 nucleotides are complementary with a sequence of the target RNA. In some embodiments, 11, 12, 13, 14, 15, 16, 17, 18, 19, 20, 21 or 22 (including the third from the 5′ end) of 22 nucleotides are complementary with a sequence of the target RNA. In some embodiments, 11, 12, 13, 14, 15, 16, 17, 18, 19, 20, 21, 22 or 23 (including the third from the 5′ end) of 23 nucleotides are complementary with a sequence of the target RNA. In some embodiments, 11, 12, 13, 14, 15, 16, 17, 18, 19, 20, 21, 22, 23 or 24 (including the third from the 5′ end) of 24 nucleotides are complementary with a sequence of the target RNA. In some embodiments, 11, 12, 13, 14, 15, 16, 17, 18, 19, 20, 21, 22, 23, 24, or 25 (including the third from the 5′ end) of 25 nucleotides are complementary with a sequence of the target RNA. In some embodiments, they are uniquely targeting siRNA sequences. In some embodiments, siRNA compounds and RISCs comprising such siRNA compounds are provided in which the siRNA sequence is a uniquely targeting siRNA sequence. In some preferred embodiments, the uniquely targeting siRNA and the RISCs comprising the same are directed against RNA encoding oncogenes, tumor suppressor genes, genes causing neurogenerative diseases, viral genes, cytokines, interleukins, chemokines, co-stimulatory molecules and growth factors. In some preferred embodiments, the uniquely targeting siRNA and the RISCs comprising the same are directed against RNA encoding p53, MYC, MYB, PTEN, HER-2/NEU, TGF, PDGFR, Ras, Rb (Retinoblastoma protein), bcl-2, bax, phosphatase 2A, telomerase, p16, p21, Cyclin Dependent Kinases (CDKs), TRAIL, TNF, Mitogen Activated Kinases (MAPKs), VHL, NfkB, Merlin, Schwanomin, Notch, APC, AXIN2, E-Cadherin, QPC3, AXT1,2. Patched, SUFU, FH, SDHB,C,D, WT-1, STK11, TSC1, TSC2, BMPR1A, SMAD4, NF2, BHD, HRPT2, MUTYH, ATM, BLM, BRCA1, BRCA2, FANCA,C,D2,E,F,G, MSH2, MLH1, MSH6, PMS2, XPA, KT, MET, RET, HIF-1, b-catenin, FAS, FBXW7, GLI, HPEVE6, MDM2, AKT2, FOXO1A,3A, PI3KCA, CYCLIN D1, HPVE7, TAL1, TFE3, ALK, ABL1, BRAF, EGFR, EPHB2, ERBB2, FES, FGFRs, FLT3,4, JAK2, N-RAS, K-RAS, PDGFB, PDGFRB, EWSR1, RUNX1, SMAD2, TGFFBR1, 2, MYCN, HMGA2, HOXA9,11,13, HOX11,11L2, MKK4, MLL, SS18, RARA, PTNP1,11, APP (Amyloid Precursor protein), Tau, synucleins, alpha-synuclein (PARK1), Parkin (PARK2), PARK5, Huntingtin, SOD (Superoxide Dismutase), Senataxin, Apolipoprotein-E, DJ-1 (PARL7), presenilin-1, presenilin-2, IL-1, IL-2, IL-4. TNF, Interferons, (IFNA, IFNB, IFN-gamma), CD4, CD8, CD19, CD5, CD43, CD1, CD3, B-cell receptor, T-cell Receptor, VEGF, bcr/abl, HIV genes such asrev, env, gag, or CMV genes.

In preferred embodiments, the siRNAs and miRNAs described, designed, synthesized and used herein are active in the inhibition of RNA translation in animals, preferably humans, canines, felines, equines, bovines, porcines, ovines, avian species, and fish. In other embodiments, the siRNAs or miRNAs described, designed, synthesized and used herein are active in the inhibition of RNA translation in plants such as corn, soybean, maize, wheat, barley, sorghum, cotton, oilseed, Arabidopsis thaliana, aspen, pines, oak, legumes, tubers, fruits such as apples, oranges, lemons, limes, pears, pineapples, grapes, tomatos, peppers, squash, cucumber, melons and vegetables such as celery, broccoli, cauliflower, spinach, and carrots.

### Example

Bacterially produced proteins were expressed in BL21-DE3 CodonPlus cells (Stratagene) and were purified according to the instructions of the manufacturers. Untagged human eIF2C2 protein, His-tagged eIF2C2 (eIF2C2-His) and a fusion protein of eIF2C2 with Glutathione-S-Transferase (GST-eIF2C2) were expressed from pTYB2 (NEB), pET28 (Novagen) and pGEX-6P2 (Amersham-Pharmacia) vectors respectively. Untagged or eIF2C2-His proteins were purified from the insoluble fraction and dialyzed gradually using decreasing amounts of Urea as per Novagen's instructions. GST-eIF2C2 protein retained solubility but was often degraded during the final purification steps which included either elution with glutathione or proteolytic release of eIF2C2 from GST with PreScission protease (Amersham-Pharmacia). All bacterially produced proteins were unable to bind synthetic RNAs corresponding either to single stranded let-7 (either with or without a 5′-phosphate), or to let-7 prepared as an siRNA duplex, and were devoid of endonuclease activity (data not shown).

Protein-G purified 8C7 mAb against eIF2C2 or non immune mouse IgG were covalently cross-linked on agarose beads using the Seize Primary Immunoprecipitation Kit (Pierce) according to the manufacturer's instructions. The beads were subsequently treated with 0.1M Glycine pH 3.0 to remove free antibody and were washed with lysis buffer (20 mM Tris-HCL, pH 7.4, 200 mM NaCl, 2.5 mM MgC12). Total cell lysate prepared from 10⁸ HeLa cells in lysis buffer supplemented with Complete Protease Inhibitors (Roche) and 10 U/ml Rnasin (Promega) was applied to the 8C7 or to the IgG beads, as described in (3). After antigen capture, the beads were washed extensively with lysis buffer containing 500 mM NaCI followed by washes in buffer D (20 mM Hepes-KOH, pH 7.4, 100 mM KCL, 0.1 mM EDTA). Captured proteins were eluted by treating the beads with 2M Urea dissolved in buffer D; the eluate was immediately dialyzed against buffer D and was concentrated using Amicon devices (Microcon). The final, concentrated eluate from the 8C7 beads contained approx. 4 μg of purified eIF2C2 protein. Proteins were resolved on NuPAGE (Invitrogen) gels and stained with Silver Stain or with SimplyBlue SafeStain (Invitrogen).

RNA was isolated from 0.5 μg (5 pmoles) of immunopurified eIF2C2 protein, fractionated on 15% denaturing polyacrylamide gel along with known amounts of synthetic let-7 RNA (SEQ ID NO:1 5′-UGAGGUAGUAGGUUGUAUAGU-3′; Dharmacon), transferred to a Hybond-N+ membrane (Amersham-Pharmacia) and hybridized with a 5′-end 32P-labeled DNA oligonucleotide probe complementary to let-7 (SEQ ID NO:2 5′-ACTATACAACCTACTACCTCA-3′).

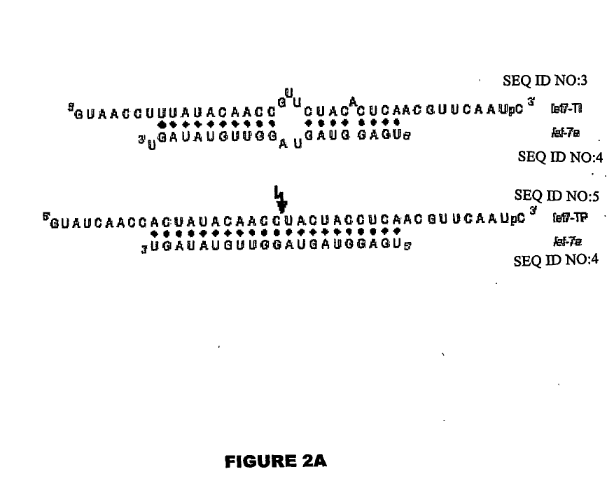

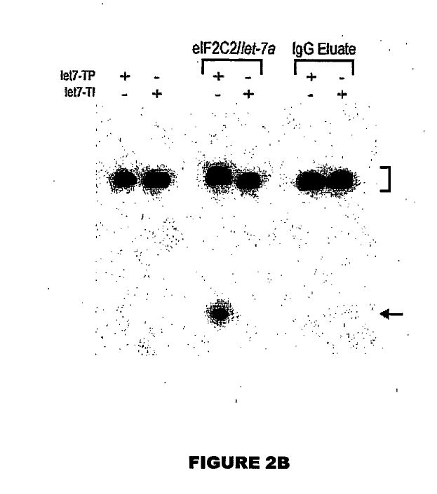

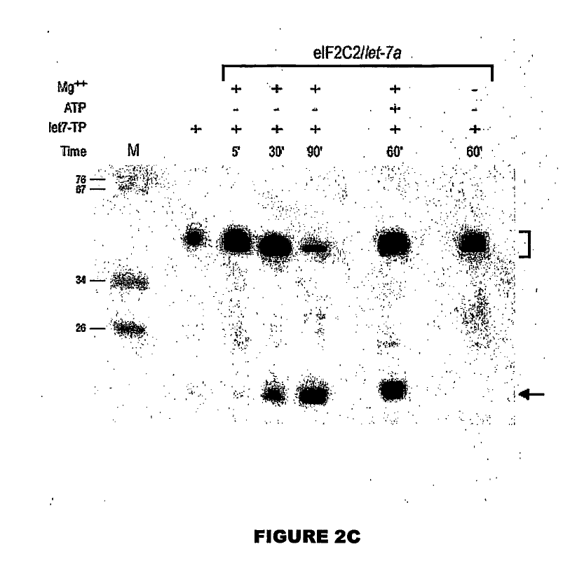

All RNAs were chemically synthesized (Dharmacon). let7-TP and let7-TI contained a 5′-Biotin and were 3′-end labeled with [5′-³²P]-pCp and T4 RNA ligase (NEB). All other RNAs were 5′-end labeled with [y-³²P]-ATP and T4 polynucleotide kinase (NEB). Cleavage assays were performed in a buffer containing 20 mM Hepes-KOH, pH 7.4, 40 mM KCL, 40 mM Potassium Acetate, 2 mM MgCl2, 1 mM DTT, 10 U/ml Rnasin, at 37° C., for 60 minutes or as indicated in the Figure legends. In some assays (FIG. 2D), 1 mM ATP was included or MgCl2, was omitted from the reactions. RNA was isolated from cleavage reactions and resolved in denaturing polyacrylamide gels.

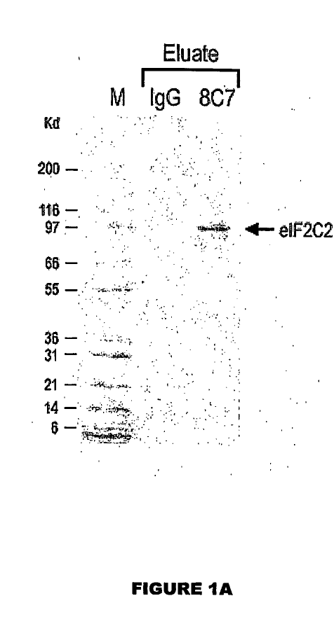

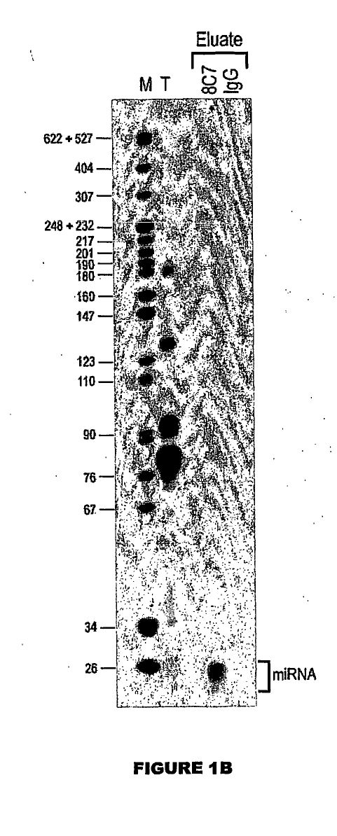

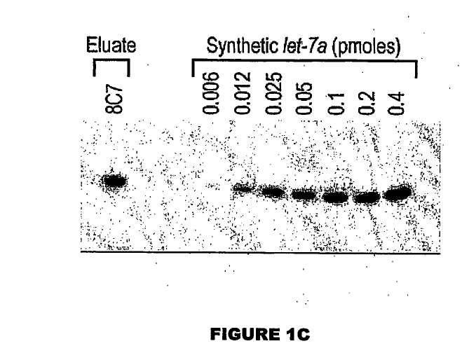

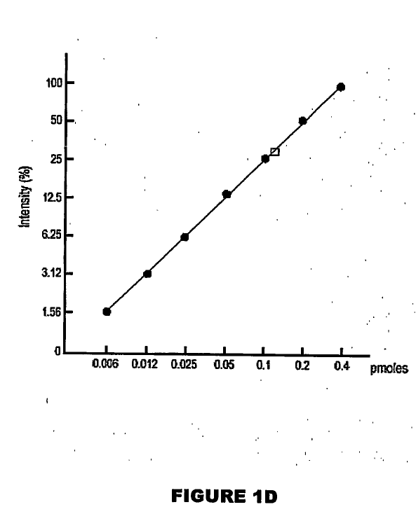

Since Argonaute proteins are essential for RNAi (22) (24) (25) and are the only protein components that miRNPs (3) and Drosophila (10) and human (26) RISCs have in common, it an Argonaute protein bound to mi/siRNAs may be the elusive miRNP/RISC endonuclease. To test this hypothesis the human eIF2C2 protein was studied because it is present both in human miRNPs (3) and human RISCs (26). Recombinant human eIF2C2 protein containing a single miRNA species was generated by expressing human eIF2C2 protein in bacteria (which do not contain miRNAs), and loading the purified, recombinant eIF2C2 protein with synthetic RNAs. However, all attempts to produce active recombinant eIF2C2 protein, using multiple systems, failed (31). eIF2C2 protein is tightly bound to miRNAs (see below) and it is very likely that proper folding and incorporation of miRNAs with elF2C2 can occur only in cells from organisms that contain miRNAs and miRNA-associated machinery. Expression of human eIF2C2 protein in a heterologous eukaryotic expression system (such as the baculovirus-based, insect-cell system) should in principle allow for proper elF2C2 folding and miRNA incorporation. This approach, however, is complicated by the presence of numerous Argonaute protein paralogs within each organism (21) that may interfere with overexpression of exogenous, human eIF2C2 protein. For this reason and because knowledge about the biogenesis of eIF2C2-miRNA ribonucleoproteins is lacking, endogenous human eIF2C2 was immunopurified from HeLa cells using monoclonal antibody (mAb) 8C7 which is described in (3), under conditions in which Dicer or the other protein components of miRNPs did not copurify (32). As shown in FIG. 1A, the 8C7 eluate contained a single protein with an apparent molecular mass of ˜95 KDa while the eluate from the negative control, non-immune mouse IgG contained no detectable proteins. Microsequencing by nanoelectrospray mass spectrometry confirmed the identity of the protein present in the 8C7 eluate as eIF2C2.

To test for the presence of miRNAs, RNA was isolated from total HeLa cell lysate, from 100 ng of immunopurified eIF2C2 and from an equivalent volume of control IgG eluate; the RNA was 3′-end-labeled with [5′-³²P]-pCp and an aliquot was analyzed by denaturing polyacrylamide electrophoresis. As shown in FIG. 1B, ˜22 nt RNAs corresponding to microRNAs are present only in the lane containing RNA from purified eIF2C2 (8C7 eluate). A much less abundant RNA band at ˜80 nt, present also in the IgG eluate, most likely represents traces of tRNA. These results show that miRNAs are stably bound to eIF2C2 and resist high salt washes (500 mM NaCl) and Urea treatment. Numerous miRNAs associate with eIF2C2 containing miRNPs as single stranded forms (3). Similarly, siRNAs are found as single stranded molecules in purified human RISCs, which also contain eIF2C2 and eIF2C1 (26). Likewise, the immunopurified eIF2C2 protein is expected to contain a heterogeneous population of miRNAs.

To calculate the amount of let-7a bound to eIF2C2 protein, quantitative Northern blot analysis was performed (33). As shown in FIG. 1C/1D, 5 pmoles (0.5 μg) of purified eIF2C2 protein contains ˜0.1 pmoles of let-7a, showing that ˜2% of purified eIF2C2 is complexed with let-7a; the remainder of the protein is most likely complexed with other miRNAs, although the possibility that a fraction of eIF2C2 protein is devoid of miRNAs cannot be exclude.

To test whether the eluted, immunopurified eIF2C2 protein had microRNA-programmed endonuclease activity, the mi/siRNA mediated, in vitro cleavage assay developed by Tuschl and colleagues (5) and Zamore and colleagues (27) was used. In this assay, perfect complementarity between an si/miRNA and its RNA target leads to endonucleolytic cleavage of the RNA target at a site corresponding to the middle of the si/miRNA complementary site, while no cleavage is seen when the complementarity between the si/miRNA and the RNA target is partial. Two target RNA oligonucleotides, one bearing a site with perfect and another with imperfect complementarity to let-7a (let7-TP and let7-TI, respectively; FIG. 2A), were 3′-end labeled with [5′-³²P]-pCp and were incubated with eluted, immunopurified eIF2C2 or IgG eluate (negative control), in the absence of ATP or other nucleotides (34). As shown in FIG. 2B, immunopurified eIF2C2 directed the endonucleolytic cleavage of let7-TP at a site corresponding to the middle of the let-7a-complementary site, but did not cleave let7-TI; the IgG eluate showed no endonuclease activity. Next tested was whether ATP or magnesium ions were required for target RNA cleavage. As shown in FIG. 2C, addition of ATP did not enhance target RNA cleavage but magnesium was required for endonucleolytic cleavage. These findings are consistent with previous studies demonstrating that target RNA cleavage does not require ATP (11), and demonstrate that in vitro, eIF2C2, an Argonaute protein, with its associated miRNAs is sufficient for both target RNA recognition and endonucleolytic cleavage. The general term mirgonaute is proposed to designate a ribonucleoprotein composed of an Argonaute protein complexed with mature miRNAs. Because of the purity of the miRNA containing eIF2C2 ribonucleoprotein (FIG. 1A/1B) used in the cleavage assays, it is unlikely that the endonuclease activity resides in another protein or RNA molecule (other that miRNAs bound to eIF2C2) that has escaped detection. The possibility cannot, however, be excluded that another small cofactor that is tightly bound to eIF2C2 or the miRNAs is also required for endonucleolytic activity.

To calculate the rate of target RNA cleavage 1 pmole (100 ng) of purified eIF2C2 protein, containing ˜0.02 pmoles of let-7a miRNA with 0.4 pmoles of radiolabeled target RNA, let7-TP was incubated for 5, 30 and 90 minutes in a cleavage assay and quantitated the amount of product generated as a function of time. As shown in FIG. 2C, 0.02 pmoles of let-7 miRNA, complexed with eIF2C2, cleaved ˜60% of the target RNA in 90 minutes, indicating that the let-7 containing eIF2C2 ribonucleoprotein catalyzed ˜12 rounds of target RNA cleavage. These results are consistent with the finding that in HeLa S100 extract miRNAs are components of multiple turnover enzymes and are in agreement with the estimated ˜10 rounds of target RNA cleavage mediated by HeLa S100 extracts (27).

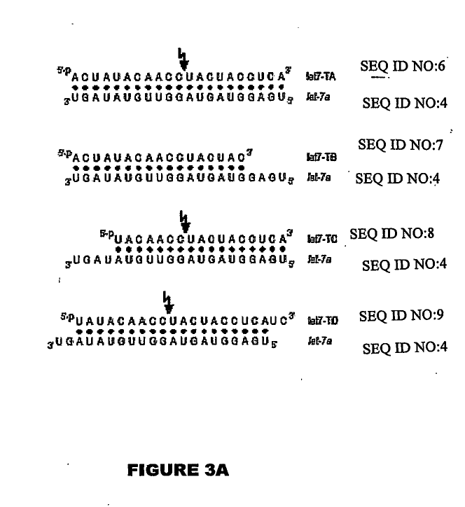

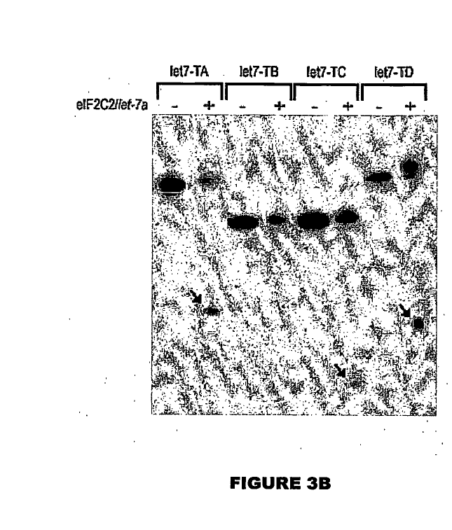

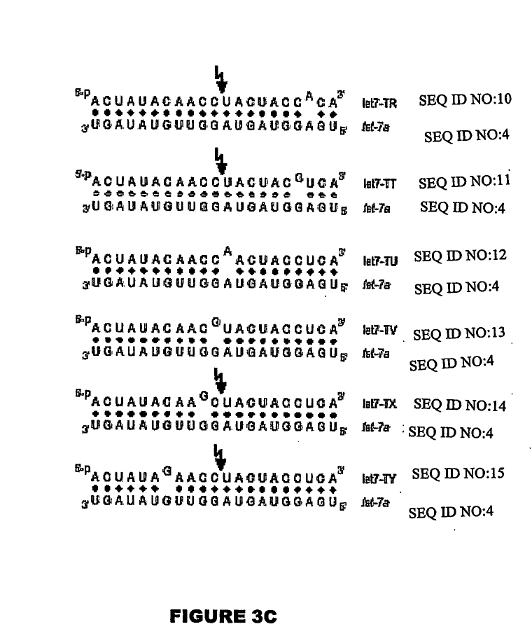

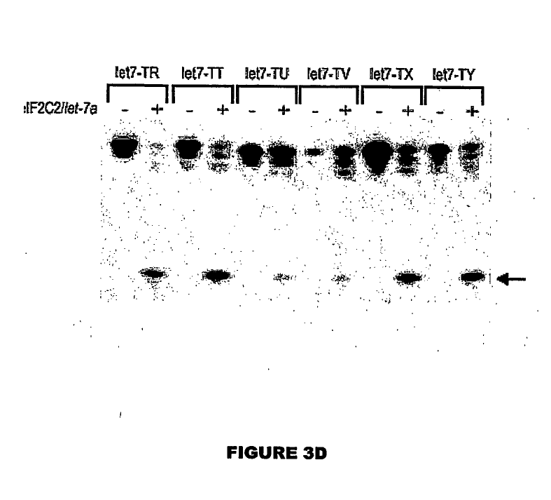

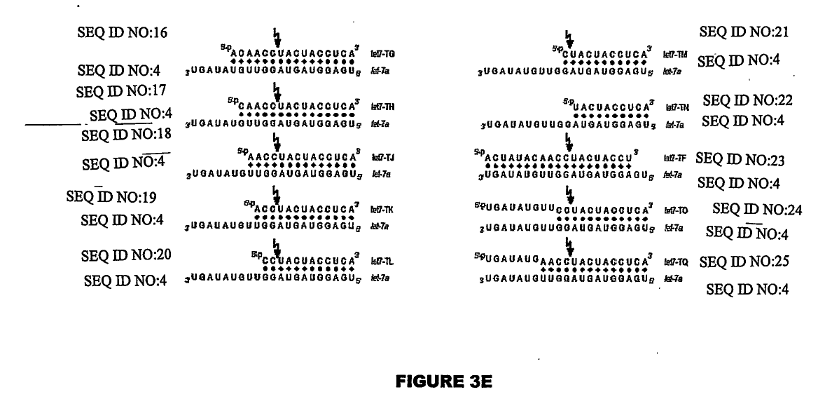

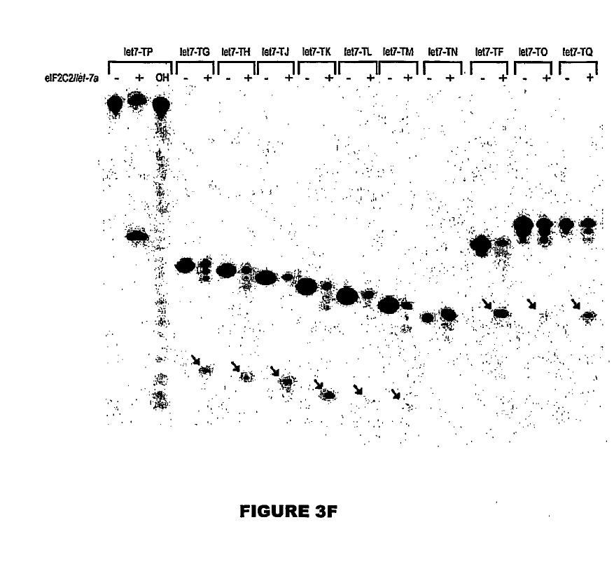

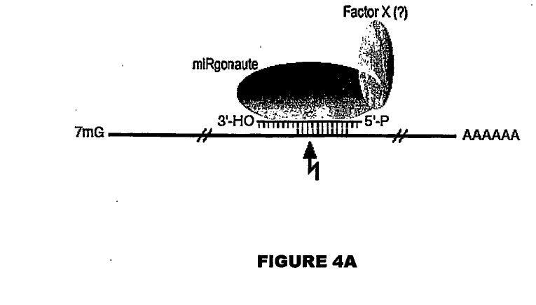

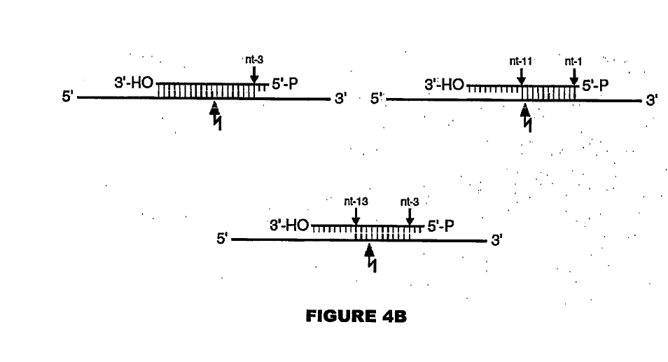

To investigate further the requirements for target RNA recognition and cleavage by purified eIF2C2/let-7, four additional RNAs were tested in cleavage assays: let7-TA, a 21-mer with perfect complementarity to let-7; let7-TB, a 17-mer that base pairs with the last 17 nucleotides (from the 5′-end) of let-7; let7-TC, a 17-mer that base pairs with the first 17 nucleotides of let-7; and let7-TD, a 21-mer that base pairs with let-7, leaving 2 nt, 3′-overhangs, and mimicking an siRNA duplex (FIG. 3A). As shown in FIG. 3B, all target RNAs, except let7-TB, were cleaved by purified eIF2C2/let-7a, at the same position, between the 10th and 11th nucleotide starting from the 5′-end of let-7a. These findings demonstrated, surprisingly, that miRgonautes may cleave RNAs that are shorter than their miRNAs and prompted us to investigate further the requirements for miRNA:target RNA recognition and cleavage. A series of RNA targets with varying binding properties with let-7a were synthesized and tested them in cleavage assays. As shown in FIG. 3C/3D, point mutations of the target RNA that disrupt base pairing with let-7a, had no effect in let7a-mediated target RNA cleavage with the exception of targets let7-TU and let7-TT. These two RNAs disrupt base pairing with nucleotides 10 and 11 of the let-7a miRNA, which have been shown to be critical for target RNA cleavage. Surprisingly, RNA targets as short as 11 nucleotides (let7-TG to let7-TM) were still cleaved by eIF2C2/let-7a, as long as they hybridized perfectly with the 5′-most half of let-7a (FIG. 3E/3F). The 10-mer let7-TN RNA was not cleaved by eIF2C2/let-7a. An RNA target (let7-TN) that does not base pair with the first 2 nucleotides of let-7a is also cleaved by EIF2C2/let-7a. (FIG. 3E/3F). These findings suggest that the first (from the 5′-end) half of an miRNA is sufficient for target RNA recognition and cleavage and that even the first two nucleotides of an miRNA are dispensable for target RNA recognition and cleavage. Two longer RNA targets that base pair with the first 12 or 14 nucleotides of let-7a (Let7-TO and let7-TQ) were also tested. As shown in FIG. 3E/3F, both of these targets were cleaved. Collectively, these findings have profound implications for miRNA function and for the use of siRNAs for knockdowns. This study shows that only a portion of an miRNA, bound to an Argonaute protein, is required for target RNA recognition and cleavage (summarized in a schematic form in FIG. 4). Up to now computational prediction of miRNA targets (for miRNAs that act as siRNAs) has relied on near perfect complementarity between the entire nucleotide sequence of an miRNA and its putative mRNA target. This strategy has successfully predicted miRNA targets for most plant miRNAs but failed to uncover targets for animal miRNAs (28). Some miRNAs may use only a portion of their sequence (as defined in this study) to recognize their targets. This is consistent with a recent report describing that only the first 13 nucleotides of the novel plant miRNA JAW, are required for target RNA recognition and cleavage in vivo (31). The second important finding that stems from this study is in the design of siRNAs for experimental knockdowns. A recent report has observed off target regulation by siRNAs, thus questioning the specificity of siRNAs (32). The critical determinant of siRNA specificity (as defined in this study) are the nucleotides shown in FIG. 4. In order to design siRNAs with high specificity, and to avoid off target effects, these nucleotides should not share homology with any mRNA but the one that is targeted.

In summary, this study defines the nucleotides of an miRNA that are critical for target RNA regulation and cleavage and demonstrates that a miRgonaute (an Argonaute protein eIF2C2 with its associated miRNA -let-7a-) is a core miRNP/RISC endonuclease. It is likely that other Argonaute proteins act as mi/siRNA programmed endonucleases. It is also possible that in vivo, other proteins may be required for optimal target RNA recognition and/or miRNP/RISC activity.

### REFERENCES

The following references are hereby incorporated herein by reference in their entirety.
1. B. J. Reinhart, D. P. Bartel, Science 297, 1831 (Sep. 13, 2002).
2. M. Lagos-Quintana, R. Rauhut, W. Lendeckel, T. Tuschl, Science 294, 853-8 (Oct. 26, 2001).
3. Z. Mourelatos et al., Genes Dev 16, 720-8 (Mar. 15, 2002).
4. A. J. Hamilton, D. C. Baulcombe, Science 286, 950-2 (Oct. 29, 1999).
5. S. M. Elbashir, J. Martinez, A. Patkaniowska, W. Lendeckel, T. Tuschl, Embo J 20, 6877-88 (Dec. 3, 2001).
6. A. Fire et al., Nature 391, 806-11 (Feb. 19, 1998).
7. R. H. Plasterk, Science 296, 1263-5 (May 17, 2002).
8. M. T. McManus, P. A. Sharp, Nat Rev Genet 3, 737-47 (October, 2002).
9. E. Bernstein, A. A. Caudy, S. M. Hammond, G. J. Hannon, Nature 409, 363-3 (Jan. 18, 2001).
10. S. M. Hammond, S. Boettcher, A. A. Caudy, R. Kobayashi, G. J. Hannon, Science 293, 1146-50 (Aug. 10, 2001).
11. A. Nykanen, B. Haley, P. D. Zamore, Cell 107, 309-21 (Nov. 2, 2001).
12. N. C. Lau, L. P. Lim, E. G. Weinstein, D. P. Bartel, Science 294, 858-62 (Oct. 26, 2001).
13. R. C. Lee, V. Ambros, Science 294, 862-4 (Oct. 26, 2001).
14. G. Hutvagner et al., Science 293, 834-8 (Aug. 3, 2001).
15. R. F. Ketting et al., Genes Dev 15, 2654-9 (Oct. 15, 2001).
16. S. W. Knight, B. L. Bass, Science 293, 2269-71 (Sep. 21, 2001).
17. R. C. Lee, R. L. Feinbaum, V. Ambros, Cell 75, 843-54 (Dec. 3, 1993).
18. B. J. Reinhart et al., Nature 403, 901-6 (Feb. 24, 2000).
19. P. H. Olsen, V. Ambros, Dev Biol 216, 671-80 (Dec. 15, 1999).
20. K. Seggerson, L. Tang, E. G. Moss, Dev Biol 243, 215-25 (Mar. 15, 2002).
21. M. A. Carmell, Z. Xuan, M. Q. Zhang, G. J. Hannon, Genes Dev 16, 2733-42 (Nov. 1, 2002).
22. H. Tabara et al., Cell 99, 123-32 (Oct. 15, 1999).
23. A. Grishok et al., Cell 106, 23-34 (Jul. 13, 2001).
24. C. Catalanotto, G. Azzalin, G. Macino, C. Cogoni, Nature 404, 245 (Mar. 16, 2000).
25. N. Doi et al., Curr Biol 13, 41-6 (Jan. 8, 2003).
26. J. Martinez, A. Patkaniowska, H. Urlaub, R. Luhrmann, T. Tuschl, Cell 110, 563-74 (Sep. 6, 2002).
27. G. Hutvagner, P. D. Zamore, Science 297, 2056-60 (Sep. 20, 2002).
28. M. W. Rhoades et al., Cell 110, 513-20 (Aug. 23, 2002).
29. C. Llave, K. D. Kasschau, M. A. Rector, J. C. Carrington, Plant Cell 14, 1605-19 (July, 2002).
30. C. Llave, Z. xie, K. D. Kasschau, J. C. Carrington, Science 297, 2053-6 (Sep. 20, 2002).
31. J. F. Palatnik, E. Allen, X. Wu, C. Schommer, R. Schwab, J. C. Carrington, D. Weigel, Nature, doi101038/nature01958 (Aug. 20, 2003)
32. A. L. Jackson, et al., Nat Biotechnol 6, 635-7 (June, 2003)

1. A method of identifying a uniquely targeting siRNA nucleotide sequence for a target mRNA sequence of a target species comprising the steps of:
comparing a database of mRNA sequences from the target species with an siRNA nucleotide sequence that consists of 18-25 including at least 11 consecutive nucleotides complementary to the target mRNA sequence to be cleaved by the siRNA nucleotides, wherein the at least 11 consecutive nucleotides complementary to the target mRNA sequence include a nucleotide that is third from an siRNA nucleotide sequence's 5′ end; and
determining if, in addition to the target mRNA sequence, one or more additional mRNA sequences in the database are complementary to an 11 consecutive nucleotide sequence of the siRNA nucleotide sequence including the third nucleotide from the 5′ end of the siRNA nucleotide,
wherein an absence of one or more additional mRNA sequences in the database that are complementary to an 11 consecutive nucleotide sequence of the siRNA nucleotide sequence including the third nucleotide from the 5′ end of the siRNA nucleotide indicates that the siRNA nucleotide sequence is a uniquely targeting siRNA nucleotide sequence.

2. A method of designing a uniquely targeting siRNA for a target mRNA molecule comprising the steps of:
a) identifying an siRNA nucleotide sequence for the target mRNA, said sequence consisting of 18-24 nucleotides including a nucleotide sequence that has 11 consecutive nucleotides, including the third nucleotide from the siRNA nucleotide sequence's 5′ end, that are complementary to an 11 nucleotide sequence that occurs on the target mRNA molecule;
comparing the siRNA nucleotide sequence with a database of mRNA sequences from the target mRNA species; and
determining if, in addition to the target mRNA sequence, one or more additional mRNA sequences in the database are complementary to an 11 consecutive nucleotide sequence of the siRNA nucleotide sequence including the third nucleotide from the 5′ end of the siRNA nucleotide,
wherein an absence of one or more additional mRNA sequences in the database that are complementary to an 11 consecutive nucleotide sequence of the siRNA nucleotide sequence including the third nucleotide from the 5′ end of the siRNA nucleotide indicates that the of the siRNA nucleotide is a uniquely targeting siRNA nucleotide sequence.

3. The method of claim 1 wherein the database of mRNA sequences is a selected from the group consisting of: NCBI database and ENSEMBL database.

4. The method of claim 1 wherein the comparing of the siRNA nucleotide sequence with the database of mRNA sequences from the target mRNA species is performed by a computer.

5. The method of claim 1 wherein the comparing of the siRNA nucleotide sequence with the database of mRNA sequences from the target mRNA species is performed by a computer using a BLAST program.

6. The method of claim 1 wherein the siRNA nucleotide sequence consists of 18, 19, 20, 21, 22, 23, 24 or 25 nucleotides.

7. (canceled)

8. (canceled)

9. (canceled)

10. The method of claim 1 wherein the target mRNA is selected from the group consisting of: an mRNA encoding an oncogene, an mRNA encoding a pathogen protein, a cytokine, a chemokine, a co-stimulatory molecule and a growth factor.

11. (canceled)

12. (canceled)

13. A method of synthesizing a uniquely targeting siRNA for a target mRNA molecule comprising the steps of:
identifying or designing uniquely targeting siRNA nucleotide sequence for the target mRNA according to claim 1; and
synthesizing an siRNA molecule having the uniquely targeting siRNA nucleotide sequence.

14. A method of inhibiting expression of a target mRNA molecule comprising the steps of:
synthesizing a uniquely targeting siRNA for a target mRNA molecule according to claim 13; and
contacting a target mRNA molecule with an RISC that comprises an siRNA molecule that comprises the uniquely targeting siRNA nucleotide sequence.

15. A method of identifying an miRNA nucleotide sequence that does not function as a siRNA nucleotide sequence for mRNA of a target species comprising the steps of:
comparing a database of mRNA sequences from the target species with an miRNA nucleotide sequence that consists of 18-24 nucleotides; and
determining if one or more mRNA sequences in the database are complementary to an 11 consecutive nucleotide sequence of the miRNA nucleotide sequence including the third nucleotide from the 5′ end of the miRNA nucleotide,
wherein an absence of one or more mRNA sequences in the database that are complementary to an 11 consecutive nucleotide sequence of the miRNA nucleotide sequence including the third nucleotide from the 5′ end of the miRNA nucleotide indicates that the miRNA nucleotide sequence does not function as a siRNA nucleotide sequence for mRNA of a target species.

16. A method of designing an miRNA nucleotide sequence that does not function as a siRNA nucleotide sequence for mRNA of a target species comprising the steps of:
identifying an miRNA nucleotide sequence that consists of 18-24 nucleotides;
comparing the miRNA nucleotide sequence with a database of mRNA sequences from the target mRNA species; and
determining if one or more mRNA sequences in the database are complementary to an 11 consecutive nucleotide sequence of the miRNA nucleotide sequence including the third nucleotide from the 5′ end of the miRNA nucleotide,
wherein an absence of one or more mRNA sequences in the database that are complementary to an 11 consecutive nucleotide sequence of the miRNA nucleotide sequence including the third nucleotide from the 5′ end of the siRNA nucleotide indicates that the miRNA nucleotide sequence does not function as a siRNA nucleotide sequence for mRNA of a target species.

17. The method of claim 15 wherein the database of mRNA sequences is selected from the group consisting of: NCBI database and ENSEMBL database.

18. The method of claim 15 wherein the comparing of the miRNA nucleotide sequence with the database of mRNA sequences from the target species is performed by a computer.

19. The method of claim 15 wherein the comparing of the miRNA nucleotide sequence with the database of mRNA sequences from the target species is performed by a computer using a BLAST program.

20. The method of 15 wherein the miRNA nucleotide sequence consists of 18, 19, 20, 21, 22, 23, 24 or 25 nucleotides.

21. (canceled)

22. (canceled)

23. (canceled)

24. The method of claim 15 wherein the miRNA inhibits translation of mRNA selected from the group consisting of: an mRNA encoding an oncogene, an mRNA encoding a pathogen protein, a cytokine, a chemokine, a co-stimulatory molecule and a growth factor.

25. (canceled)

26. (canceled)

27. A method of synthesizing an miRNA that does not function as a siRNA nucleotide sequence for mRNA of a target species comprising the steps of:
identifying or designing an miRNA nucleotide sequence that does not function as an siRNA nucleotide sequence for mRNA of a target species according to 15; and
synthesizing an miRNA molecule that has an miRNA nucleotide sequence that does not function as an siRNA nucleotide sequence for mRNA of a target species

28. A method of inhibiting expression of a target mRNA molecule comprising the steps of:
synthesizing an miRNA that does not function as a siRNA nucleotide sequence for mRNA of a target species according to claim 27; and
contacting a target mRNA molecule with an miRNP that comprises an miRNA that does not function as a siRNA nucleotide sequence for mRNA of a target species.

29. A uniquely targeting siRNA molecule consisting of 18-25 nucleotides including a nucleotide sequence that has at least 11 consecutive nucleotides, including the third nucleotide from the 5′ end of the siRNA molecule, that are complementary to a nucleotide sequence that occurs on a target mRNA molecule and not on another mRNA from a target species.

30. The uniquely targeting siRNA molecule of claim 29 wherein the nucleotide sequence of the siRNA molecule is fully complementary with a sequence of the target mRNA molecule.

31. An siRNA molecule consisting of 18-25 nucleotides including a nucleotide sequence that has at least 11 consecutive nucleotides, including the third nucleotide from the 5′ end of the siRNA molecule, that are complementary to a nucleotide sequence that occurs on a target mRNA molecule and not on another mRNA from a target species, wherein the siRNA molecule is not fully complementary with a sequence of the target mRNA molecule.

32. The siRNA molecule of claim 31 consisting of 18, 19, 20, 21, 22, 23, 24 or 25 nucleotides.

33. (canceled)

34. (canceled)

35. (canceled)

36. The siRNA molecule of claim 29 wherein the target mRNA is selected from the group consisting of an mRNA encoding: an oncoprotein, a tumor suppressor, a pathogen protein, a cytokine, an interleukin, a chemokine, a co-stimulatory molecule and a growth factor.

37. (canceled)

38. (canceled)

39. A RISC comprising an siRNA molecule of claim 29.

40. An miRNA molecule for a target mRNA of a target species, wherein said miRNA molecule consists of 18-25 nucleotides and does not function as a siRNA nucleotide sequence for mRNA of a target species.

41. The miRNA molecule of claim 40 consisting of 18, 19, 20, 21, 22, 23, 24 or 25 nucleotides.

42. (canceled)

43. (canceled)

44. (canceled)

45. The miRNA molecule of claim 40 wherein the target mRNA is selected from the group consisting of an mRNA encoding: an oncoprotein, a tumor suppressor, a pathogen protein, a cytokine, an interleukin, a chemokine, a co-stimulatory molecule and a growth factor.

46. (canceled)

47. (canceled)


SEQUENCE LISTING (25 sequences)

SEQ ID NO: 1 - RNA, 21 nt, Artificial Sequence
  Synthetic RNA 1
ugagguaguagguuguauagu

SEQ ID NO: 2 - DNA, 21 bp, Artificial Sequence
  Synthetic DNA 1
actatacaacctactacctca

SEQ ID NO: 3 - RNA, 39 nt, Artificial Sequence
  Synthetic RNA 2
guaaccuuuauacaaccguucuacacucaacguucaauc

SEQ ID NO: 4 - RNA, 21 nt, Homo sapiens
ugauauguuggaugauggagu

SEQ ID NO: 5 - RNA, 40 nt, Artificial Sequence
  Synthetic RNA 3
guaucaaccacuauacaaccuacuaccucaacguucaauc

SEQ ID NO: 6 - RNA, 21 nt, Artificial Sequence
  Synthetic RNA 4
acuauacaaccuacuaccuca

SEQ ID NO: 7 - RNA, 17 nt, Artificial Sequence
  Synthetic RNA 5
acuauacaaccuacuac

SEQ ID NO: 8 - RNA, 17 nt, Artificial Sequence
  Synthetic RNA 6
uacaaccuacuaccuca

SEQ ID NO: 9 - RNA, 21 nt, Artificial Sequence
  Synthetic RNA 7
uauacaaccuacuaccucauc

SEQ ID NO: 10 - RNA, 21 nt, Artificial Sequence
  Synthetic RNA 8
acuauacaaccuacuaccaca

SEQ ID NO: 11 - RNA, 21 nt, Artificial Sequence
  Synthetic RNA 9
acuauacaaccuacuacguca

SEQ ID NO: 12 - RNA, 21 nt, Artificial Sequence
  Synthetic RNA 10
acuauacaaccaacuaccuca

SEQ ID NO: 13 - RNA, 21 nt, Artificial Sequence
  Synthetic RNA 11
acuauacaacguacuaccuca

SEQ ID NO: 14 - RNA, 21 nt, Artificial Sequence
  Synthetic RNA 12
acuauacaagcuacuaccuca

SEQ ID NO: 15 - RNA, 21 nt, Artificial Sequence
  Synthetic RNA 13
acuauagaaccuacuaccuca

SEQ ID NO: 16 - RNA, 16 nt, Artificial Sequence
  Synthetic RNA 14
acaaccuacuaccuca

SEQ ID NO: 17 - RNA, 15 nt, Artificial Sequence
  Synthetic RNA 15
caaccuacuaccuca

SEQ ID NO: 18 - RNA, 14 nt, Artificial Sequence
  Synthetic RNA 16
aaccuacuaccuca

SEQ ID NO: 19 - RNA, 13 nt, Artificial Sequence
  Synthetic RNA 17
accuacuaccuca

SEQ ID NO: 20 - RNA, 12 nt, Artificial Sequence
  Synthetic RNA 18
ccuacuaccuca

SEQ ID NO: 21 - RNA, 11 nt, Artificial Sequence
  Synthetic RNA 19
cuacuaccuca

SEQ ID NO: 22 - RNA, 10 nt, Artificial Sequence
  Synthetic RNA 20
uacuaccuca

SEQ ID NO: 23 - RNA, 19 nt, Artificial Sequence
  Synthetic RNA 21
acuauacaaccuacuaccu

SEQ ID NO: 24 - RNA, 21 nt, Artificial Sequence
  Synthetic RNA 22
ugauauguuccuacuaccuca

SEQ ID NO: 25 - RNA, 21 nt, Artificial Sequence
  Synthetic RNA 23
ugauaugaaccuacuaccuca